(19) United States
(12) Patent Application Publication
(10) Pub. No.: US 2014/0316330 A1
(43) Pub. Date: Oct. 23, 2014

(54) PRESSURIZED OXYGEN DELIVERY SYSTEM

(71) Applicants: Gary Fudem, Chilmurk, MA (US); Raymond Dunn, Shrewsbury, MA (US)

(72) Inventors: Gary Fudem, Chilmurk, MA (US); Raymond Dunn, Shrewsbury, MA (US)

(21) Appl. No.: 14/212,744

(22) Filed: Mar. 14, 2014

Related U.S. Application Data
(60) Provisional application No. 61/793,666, filed on Mar. 15, 2013.

(51) Int. Cl. A61M 1/00; A61M 5/14

(52) U.S. Cl. CPC A61M 1/0084; A61M 5/14; A61M 1/0088; A61M 2005/006
USPC 604/23

(57) ABSTRACT

The present invention relates to a negative pressure wound treatment system and methods for using such a system. Preferred embodiments of the invention facilitate treatment of the wound by delivering oxygen into the tissue in conjunction with the application of negative pressure.

### CROSS REFERENCE TO RELATED APPLICATION

The present application claims priority to U.S. application Ser. No. 61/793,666, filed Mar. 15, 2013, the entire contents of this application being incorporated herein by reference.

### BACKGROUND OF THE INVENTION

Negative pressure devices have been developed for the treatment of wounds. Negative pressure wound treatment utilizes devices that remove wound fluids by applying negative pressure suction to the wound. Negative pressures promote wound healing by facilitating the formation of granulation tissue at the wound site and assisting the body's inflammatory process while simultaneously removing excess fluid.

Existing methods involve the placement of foam into the wound, applying a covering over the wound to seal the wound area so that suction be applied with an external pump or vacuum source. However, further improvements in negative pressure wound therapy are needed to fully realize the benefits of treatment.

### SUMMARY OF THE INVENTION

The present invention relates to a pressure wound treatment device that provides for the introduction of oxygen into the wound in conjunction with pressure treatment. The device operates to reduce the need for repetitive replacement of wound filler material currently employed and can advance the rate of healing. The device simultaneously uses negative pressure, for example, to remove wound fluids and to deliver oxygen into the wound to facilitate healing. The device can be used to deliver additional treatment media to a wound including medications such as topical antibiotics.

In a preferred embodiment, a pressure wound treatment device includes a wound oxygen delivery material that is sized and shaped to fit within a wound opening or on a tissue surface and to deliver oxygen to the tissue. With application of a negative pressure to the delivery material, fluid removal and oxygen delivery can be provided in a single procedure. By providing for the controlled oxygen delivery into the tissue during the healing process in conjunction with the drainage of fluids from wounds as described in connection with the present invention, a substantial improvement in the rate of healing can be realized.

A negative pressure source, such as a vacuum pump, is coupled to the wound treatment material to provide negative pressure to the wound. The wound treatment material generally comprises a cellular matrix containing oxygen and/or other media, such as one or more medications, to the tissue. The device can include channels extending through the cellular matrix to provide for the removal of fluid from the tissue. A tubing system comprising one or more tubes can connect a positive and/or a negative pressure source to the delivery material. A process of cycling levels of negative pressure, or alternating positive and negative pressure can be used, for example.

The wound treatment device can be used to treat wounds or tissue on a human or animal body in which negative pressure can assist with treatment including post-surgical treatment, abdominal wounds, pressure ulcers and for wounds in the extremities (arms or legs) etc. The wound treatment device can also be used to treat wounds of different shapes, such as circular, square, rectangular or irregularly shaped wounds. A plurality of wound treatment elements can be shaped to fit within a wound and can work in combination to treat the wound. The different elements can comprise different materials including a cellular matrix and a foam filler, and can have different characteristics, such as pore size distribution, to form a composite structure.

In another preferred embodiment, the device can be configured for use in conjunction with a surgical drain. In this embodiment, one or more drain tubes can be inserted with the matrix.

### BRIEF DESCRIPTION OF THE DRAWINGS

Other features and advantages of the present invention will be apparent from the following detailed description of the invention, taken in conjunction with the accompanying drawings of which:

FIG. 1A is a perspective schematic view of a negative pressure wound treatment system.

FIG. 1B is a cross-section view of the wound treatment system.

FIG. 2 perspective view of an oxygen delivery material in accordance with the invention.

FIG. 3A illustrates a cross-sectional view of a composite wound treatment device for delivering treatment media to a wound in accordance with preferred embodiments of the invention.

FIG. 3B illustrates a surgical drain device for delivering treatment media to a surgical site.

FIG. 4 illustrates a method for treating a wound in accordance with preferred embodiments of the invention.

FIG. 5 illustrates a two-stage negative pressure wound treatment and negative pressure wound process.

FIG. 6 illustrates a control and sensor system used to control and monitor device operation in accordance with preferred embodiments of the invention.

### DETAILED DESCRIPTION OF THE INVENTION

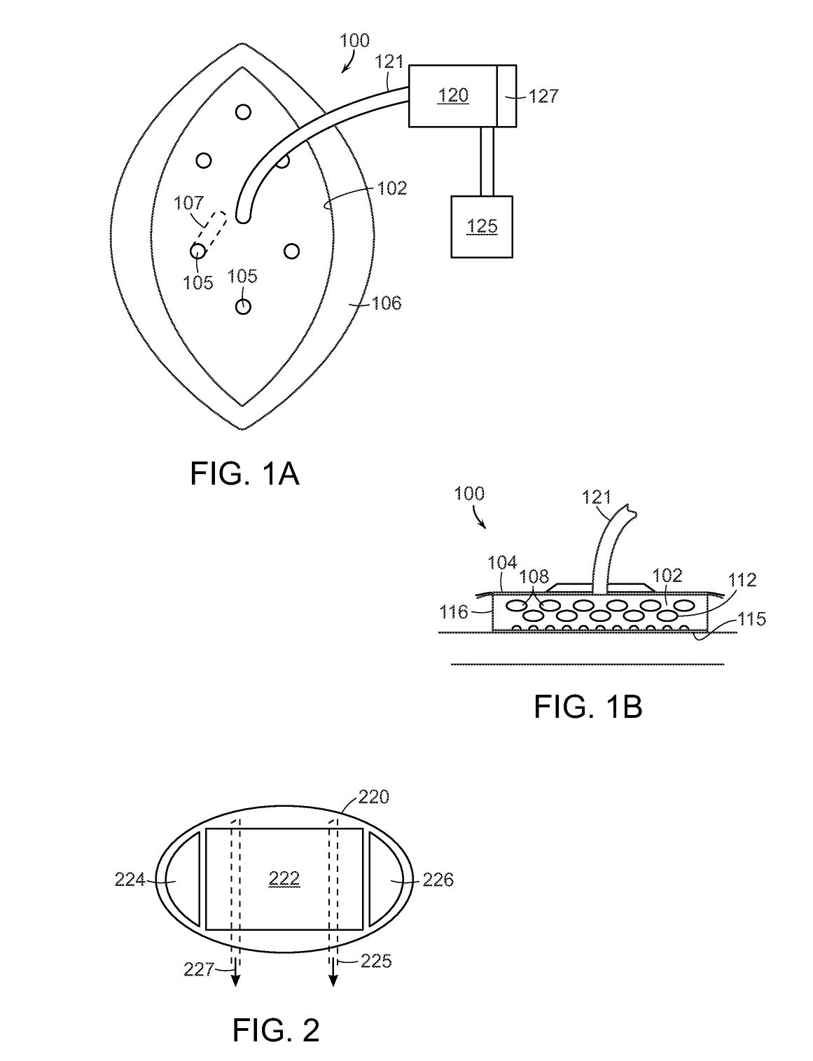

The present invention relates to a negative pressure oxygen delivery system for treating wounds. FIGS. 1A and 1B illustrate an embodiment of a wound treatment device 100 of the present invention. The device 100 includes an oxygen delivery material 102 that is sized and shaped to fit within a wound opening of a human or animal patient. In preferred embodiments, the oxygen delivery material 102 is a porous, biocompatible material, such as a closed cell polymer. By applying a negative pressure to the material 102, oxygen can be delivered into the wound while at the same time removing fluid from the wound.

Extending over at least one surface of the material 102, and preferably extending over an outer perimeter surface of the material 102 is a filler material 106. In one embodiment, the outer surface 104 is a flexible covering, such as a mesh film, that is secured to the outer perimeter surface of the device 102.

Returning to FIGS. 1A-1B, a negative pressure source 120, such as a pump, is coupled to the material 102 by a suitable coupling or conduit, such as tube 121. Additional tubes 107 can also be connected through an array of spaced ports 105 in order to spatially distribute the suction force so as to provide for both oxygen delivery and fluid removal as described herein. The negative pressure source 120 can be activated to apply a negative pressure to the material 102. The source 120 can be connected to a controller 127 that can be programmed to apply negative pressure to the wound and the oxygen delivery device. A drain container 125 can be used to store fluid from the wound. In general, the negative pressure causes a resulting pressure differential which causes fluid to be removed from underlying tissue. For example, in the embodiment of FIG. 1A, the material 102 includes a length and width dimension along the y- and x-axes, respectively, and a height along the z-axis. In order to efficiently transmit the negative pressure to the subcutaneous or other wound margins, it is preferred that the material 102 accommodate fluid removal and also operate to deliver oxygen contained therein to the tissue. It will be understood that in some embodiments, the plane of the wound margins can be curved, such as when the wound goes around the curve of an abdomen or leg.

A preferred embodiment of the invention employs an cellular structure in which one or more cells comprise hollow cavities 115. The hollow cavities can be used to store oxygen for delivery. The oxygen or other treatment media can be contained in a fluid. More details regarding the fabrication of an oxygen delivery matrix can be found in U.S. Pat. No. 7,160,553, the entire contents of this patent being incorporated herein by reference. Oxygen delivery matrix materials are available from Acrymed, Portland, Oreg. These materials comprise a cellular matrix in which a fluid can be inserted that contains dissolved oxygen and other media. This media can be formed into a device having channels extending therethrough with a patterned mold, for example. Portions of this channeled membrane can be coated with a non-permeable layer so that only surfaces in contact with skin or other tissue are sufficiently porous to enable transport of the media contained in the cells to move into the skin or other tissue. A catalyst or other trigger can be used to initiate transport of media into the tissue.

The use of hollow cells in the structure can also be used for the delivery of medication or other media into the wound. The cells 108, 112 can contain media upon implant into the wound that is subsequently released into the wound, or can be connected to an external source that provides additional material into the cellular matrix for delivery. The cell walls can have pores that open to accommodate fluid flow into the wound from within cavities therein. The location of cells can be selectively positioned within the structure depending on the preferred delivery location. For example, the cells 108 along the lateral walls can be used for delivery to the lateral tissue regions. Alternatively, the cells in the bottom plane of the device can be used for delivery to the underlying tissue structure or organs.

FIG. 1B shows the bottom of the wound treatment device 100 according to a preferred embodiment. The device 100 in this embodiment includes a smooth bottom surface 115. This material can be biocompatible film to be used with, such as, provided in conjunction with the Renasys® system available from Smith & Nephew. The bottom surface 115 provides a porous interface between the wound treatment device 100 and the underlying tissue. In the case of an abdominal wound, for example, the underlying tissue can include internal organs, such as the intestines. The smooth bottom surface 115 enables the material 102 to move without interference from the underlying tissue, and without damaging the underlying tissue. In a preferred embodiment, the bottom surface 115 includes micropores 116 (shown with size exaggerated in FIG. 1B for purposes of illustration) that allow the passage of fluid through the bottom surface 115 and into the device 100 for removal from the wound site. The wound treatment device can also be inserted over a separate layer of material so that the device will contract on top of the sliding layer. Further details concerning negative pressure wound treatment are described in U.S. application Ser. No. 13/365,615, filed Feb. 3, 2012, the entire contents of this application being incorporated herein by reference.

In some embodiments, the micropores 116 can have different sizes in different regions and/or can have different pore densities in different regions in order to direct different force levels of the vacuum source to different regions of the device 100. Similarly, the material 102 can be configured with different internal pore sizes and/or pore densities to direct the distribution of forces from the vacuum source to different areas of the device 100.

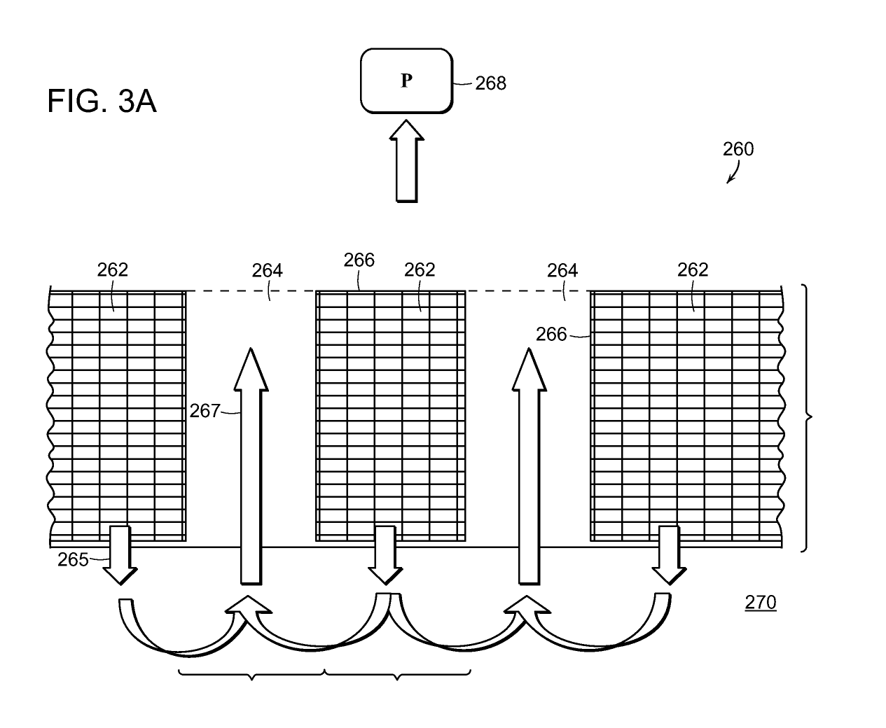

Shown in FIG. 2 is a shaped wound 220 in which a plurality of wound treatment elements are used in combination to fill the wound. In FIG. 2, elements 222, 224, 226 have different shapes that are cut or trimmed to size so as to substantially fill the wound that in this example, is oval in shape. The device can include drain tubes 225 to enable removal of fluids 227 from the wound 220 as described in greater detail herein. These tubes can be positioned above, within or under elements 222, 224, 226 to apply pressure as described herein to facilitate the delivery of media for treatment and the removal of excess fluid.

The wound closure device 200 can remain in this placed configuration for a period of several days or weeks to facilitate closing and healing of the wound 200. After a period of healing, the device 222-226 can be removed and optionally replaced with a second device such as when the treatment media has been depleted from elements 222-226. Alternatively, a needle can be inserted into the material to replenish the media being delivered. After the wound has been sufficiently treated using the present device, it can be stitched closed.

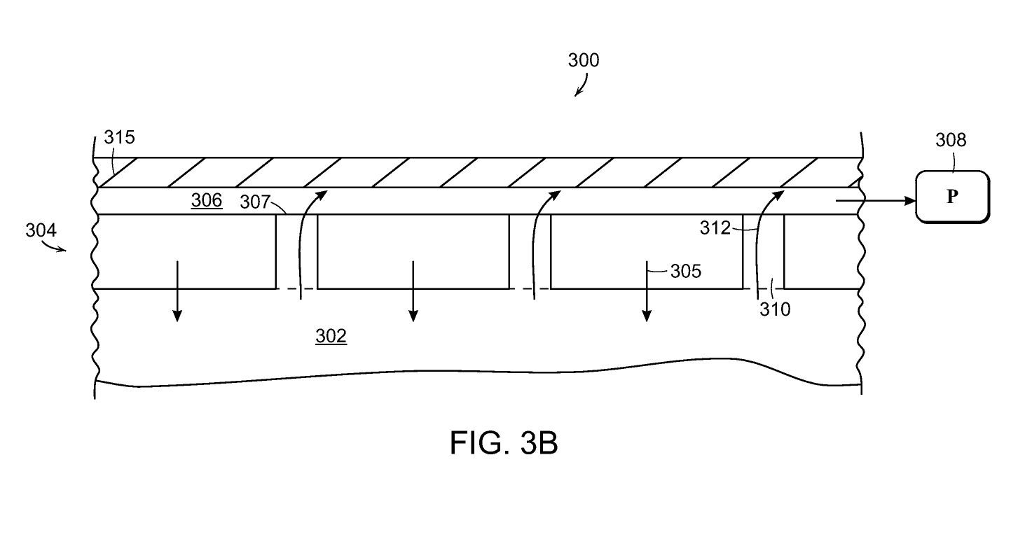

Shown in FIG. 3A is a preferred embodiment of a composite structure for a wound treatment device 260 in accordance with the invention. In this embodiment, the device 260 can comprise an array of oxygen containing cells 262 that are interspersed with channels 264 that can contain filler material. In this embodiment, a negative pressure source 268 draws a flow 265 through the filler regions 264 which also operate to draw oxygen from regions 262 into the tissue 270 underlying the device 260. The walls 266 of the cell region 262 that are exposed to negative pressure are non-porous and thus will not allow leakage in other directions. Fluid is then removed 267 by flowing through the channels 264. The pressure source can also be cycled between positive and negative pressure to facilitate both the delivery of media from the cellular matrix into the tissue and the removal of exudate fluid from the wound 270. Medications and other wound treatment therapies can also be delivered using the present device.

Shown in FIG. 3B is an embodiment 300 using a matrix 304 as described herein in which the matrix is placed in intimate contact with a wound surface 302 in which overlying tissue 315 envelops the device 304. One or more drainage tube(s) 306 can be positioned adjacent to the matrix in which apertures 307 are formed allowing the flow 312 of fluid through channels 310 in the matrix. The tube(s) 306 are connected to a negative pressure source 308 to cause removal of the fluid. Alternatively, a second tube(s) can be placed within or underneath device 304 wherein the upper tube is connected to a pressure source to apply a positive pressure and the lower tube is used to apply a negative pressure.

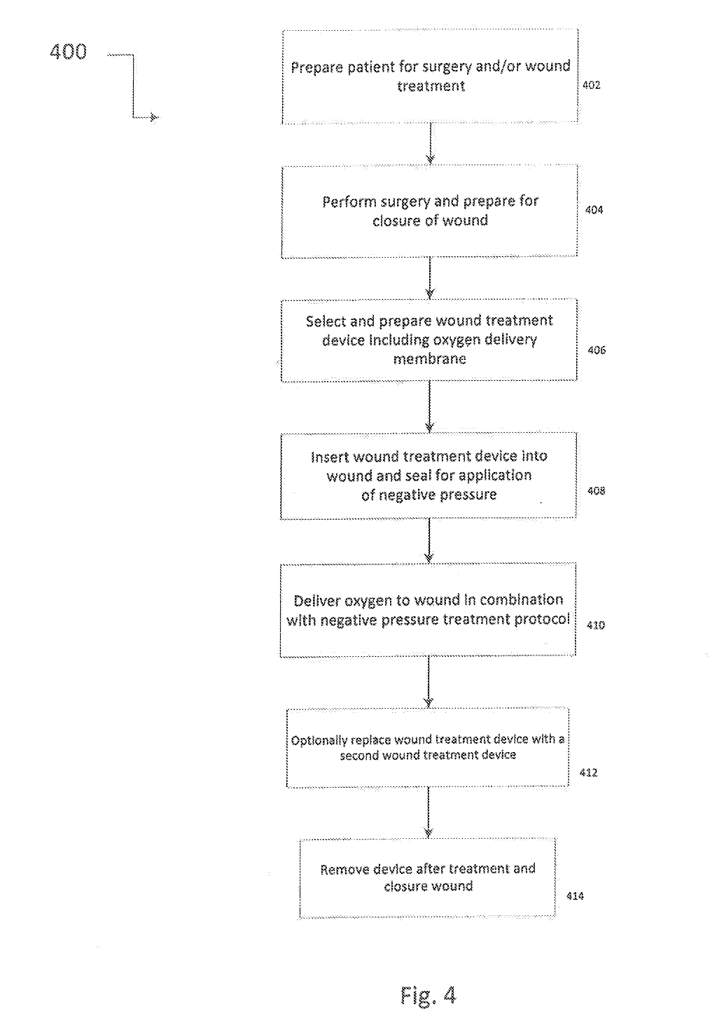

A method of performing a surgical procedure 400 using a wound closure device in accordance with preferred embodiments of the invention as illustrated in FIG. 4. After preparation 402 of the patient for surgery, an incision is made 404 to expose the surgical site, typically in the abdomen. After the procedure is performed, the wound is prepared 404 for closure. The proper size and shape of the wound treatment device is selected 406 with the peripheral tissue attachment members positioned around the circumference or outer wall surface of the device. The device is inserted 408 into the wound and the film and negative pressure system is attached 410. Negative pressure is then applied 412 to exert a suction force on the wound. Depending on the particular application, large wounds may require placement 412 of a smaller second device after removal of the first larger device. Finally, the device is removed 414 and the wound is closed, typically by suturing.

Certain types of wounds that can be treated with negative pressure wound therapy involve the separation by incision of subcutaneous tissue to form a wound opening. This procedure is frequently used to access underlying structures, organs or injuries. Additionally, many chronic, non-healing wounds such as pressure sores, vascular insufficiency or ischemic conditions and tissues undergoing radiation therapy that is used for treating cancer, for example, can utilize the devices and methods described herein.

The flow rate from the drain tubes can be regulated by flow control elements. The flow rate can also be measured or the pressure of fluids can be measured by ultrasound devices or by other imaging devices or methods. The system can also be used in conjunction with wound dressings that can also be attached to a negative pressure source to remove fluids from the wound.

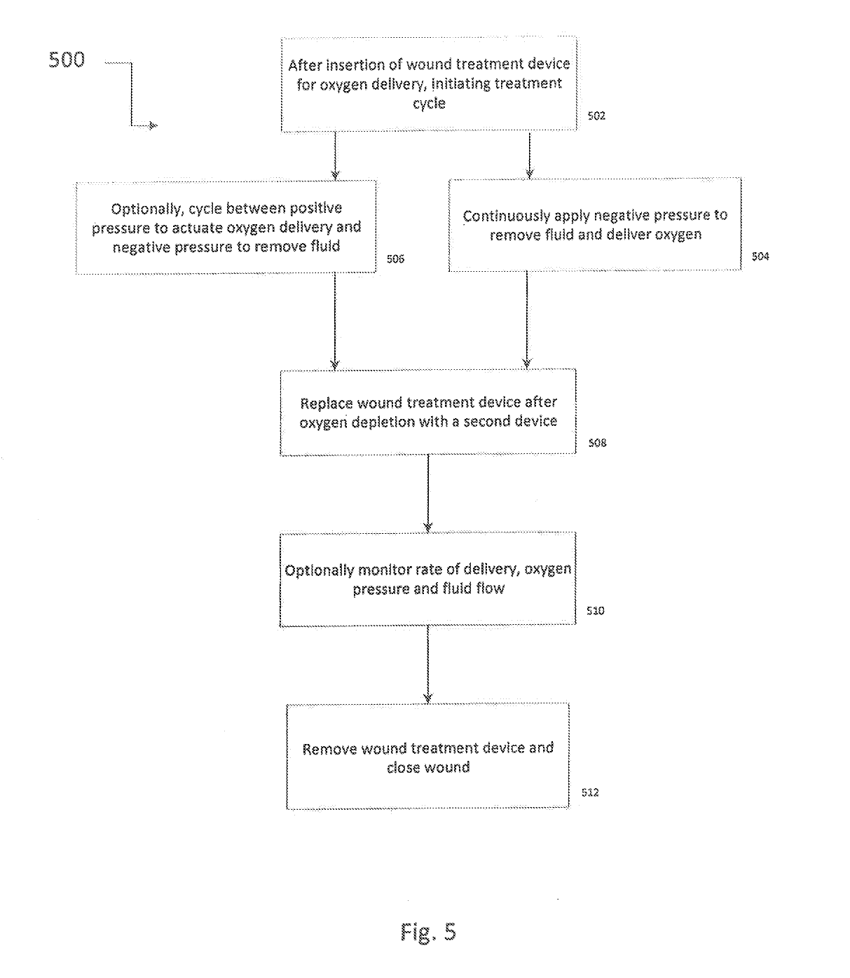

Illustrated in FIG. 5 is a further details of preferred methods 500 for treating a wound using a negative pressure oxygen delivery system. For example, as the level of negative pressure applied to wound treatment device 102 is increased. The rate of oxygen delivery can also be increased, thus providing a method of regulating the rate of delivery. Thus, after a wound treatment device is inserted 502 into the wound, negative pressure can be applied continuously 504 to simultaneously deliver oxygen and remove fluid. Alternatively, a user can apply a positive pressure to the oxygen deliver device to deliver oxygen and then switch to the application of negative pressure to remove fluid, thereby providing for sequential application 506 of the two treatment modes. After depletion of oxygen from the device, it can be replaced 508 as needed to continue treatment. The process can be monitored by imaging and/or sensor techniques to monitor and control the rate of oxygen delivery and fluid removal.

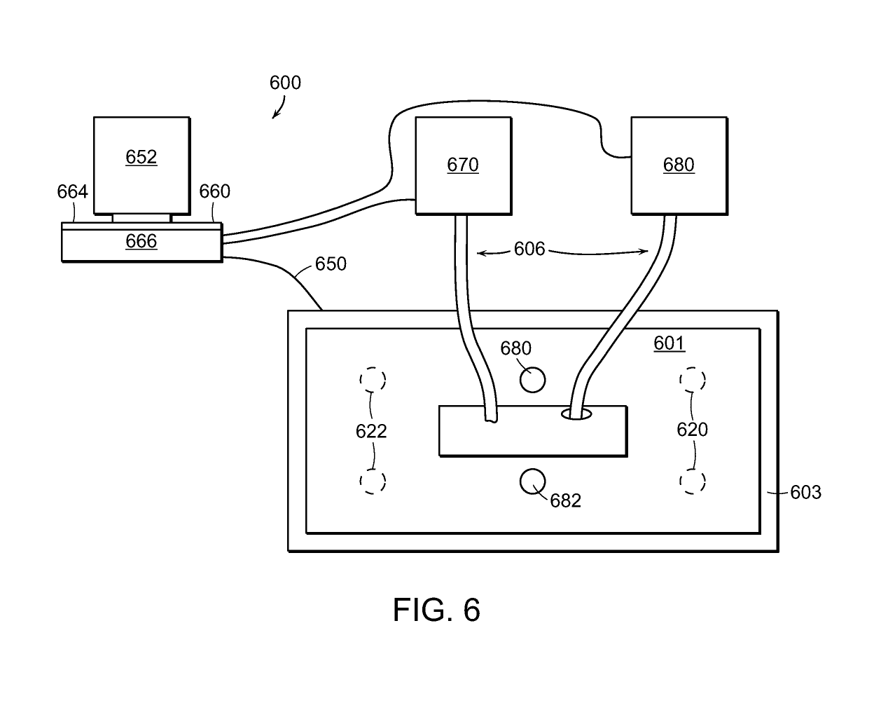

Shown in FIG. 6 is a pressure sensor system positioned to measure the pressure on underlying tissue. The sensor elements 620, 622 can measure pressure at the sliding interface 603 or at the bottom of panel 601, which can measure the amount of negative pressure at the tissue interface such as in the abdominal cavity. This can be used to monitor pressure on the tissue that can arise during the application of pressure to the wound.

The systems in FIG. 6 can optionally include a feedback control system 600 that controls a level and/or distribution of negative and/or positive pressure within the system. Both a positive pressure source 670 and a negative pressure source 680 can be connected to device 601 and the control system 600 can be programmed to execute a pressure cycling sequence to facilitate delivery of media and the removal of exudate from the wound. Sensors 680, 682 can be connected to processor housing 660 using cable 650 and pressure sensors 680 can measure fluid pressure such that sensor data are transmitted to processor housing 660. Sensors including devices and imaging methods can also be used to monitor the delivery of oxygen or other media to the tissue. Thus oxygen sensors can be deployed in an array to monitor oxygenation of the tissue, for example. A data processor 666 can be programmed to adjust the applied pressure via tubes 606, for example, to prevent injury to the patient and optimize the rate of oxygen delivery and fluid removal to improve wound healing. Data can be displayed on display 662 and a control panel 664 provides a user interface for operation of the system.

While the invention has been described in connection with specific methods and apparatus, those skilled in the art will recognize other equivalents to the specific embodiments herein. It is to be understood that the description is by way of example and not as a limitation to the scope of the invention and these equivalents are intended to be encompassed by the claims set forth below.

1. A pressure wound treatment device, comprising:
a wound treatment device adapted to be positioned on a wound surface, the wound treatment device containing oxygen that is delivered to the wound; and
a pressure delivery device that applies a fluid pressure to the wound treatment device.

2. The wound treatment device of claim 1 further comprising a filler material to be positioned in a wound opening.

3. The wound treatment device of claim 1, further comprising a negative pressure source that is coupled to the wound filler material.

4. The wound treatment device of claim 2, wherein the wound filler material comprises a porous material.

5. The wound closure device of claim 2, wherein the wound filler material comprises a foam.

6. The wound treatment device of claim 2, wherein the wound treatment device includes the wound filler material.

7. The wound treatment device of claim 1, further comprising a film that is provided over a surface of the wound treatment device.

8. The wound treatment device of claim 7, wherein the wound treatment device further comprises a mesh material.

9. The wound treatment device of claim 1, wherein the wound treatment device includes a plurality of cells and a plurality of channels that extend through the device.

10. The wound treatment device of claim 1, wherein the wound treatment device comprises a cellular matrix.

11. The wound treatment device of claim 9, wherein the wound treatment device comprises one or more layers of cells.

12. The wound treatment device of claim 1, wherein the material has a size including a length, a width and a height dimension.

13. The wound treatment device of claim 1 wherein the wound treatment device comprises a porous polymer material having a plurality of cells, each cell containing oxygen.

14. The wound treatment device of claim 1 wherein the negative pressure source is connected to a controller to apply different pressure levels to the device and the wound as described herein.

15. A method of treating a tissue surface comprising:
inserting a tissue treatment device on a region of tissue, the tissue treatment device comprising an oxygen delivery material that deliveries oxygen to the wound; and
applying a pressure to the tissue treatment device with a fluid pressure delivery device.

16. The method of claim 15, wherein the tissue comprises a wound.

17. The method of claim 15, wherein the wound comprises an ischemic wound.

18. The method of claim 15, wherein the wound comprises a chronic wound.

19. The method of claim 15 further comprising applying negative pressure to an abdominal wound.

20. The method of claim 15, further comprising removing fluid from the wound using a fluid management system.

21. The method of claim 20, wherein the fluid management system comprises a drain tube attached to a fluid container.

22. The method of claim 15 further comprising delivering oxygen to the wound with the wound treatment device, the wound treatment device comprising a polymer matrix of cells, each cell containing oxygen.

23. The method of claim 15 further comprising actuating the negative pressure source with a controller.

24. The method of claim 15 further comprising inserting the treatment device into a wound opening between margins of the wound.
